(19) United States
(12) Patent Application Publication
(10) Pub. No.: US 2012/0016042 A1
(43) Pub. Date: Jan. 19, 2012

(54) COBALT-BASED CATALYST ON A SILICA-ALUMINA SUPPORT FOR FISCHER-TROPSCH SYNTHESIS

(75) Inventors: Sylvie Maury, Charly (FR); Christele Legens, Lyon (FR); Loic Sorbier, Seyssuel (FR); Fabrice Diehl, Lyon (FR); Joseph Lopez, Saint Julien Les Rosiers (FR); Lars Fischer, Vienne (FR)

(73) Assignees: IFP Energies nouvelles, Rueil-Malmaison Cedex (FR); ENI S.p.A., Roma (IT)

(21) Appl. No.: 13/180,733

(22) Filed: Jul. 12, 2011

(30) Foreign Application Priority Data
Jul. 16, 2010 (FR) 10/03.006

(51) Int. Cl. C07C 1/04; B01J 23/755; B01J 23/745; B01J 23/46; B01J 21/04; B01J 21/06; B01J 23/83; B01J 23/63; B01J 21/10; B01J 23/89; B01J 23/882; B01J 23/883; B01J 23/881; B01J 23/652; B01J 23/847; B01J 23/889; B01J 23/656; B01J 23/75

(52) U.S. Cl. 518/721; 502/325; 502/337; 502/338; 502/332; 502/335; 502/336; 502/260; 502/259; 502/258; 502/261; 502/304; 502/328; 502/326; 502/313; 502/315; 502/316; 502/339; 518/715; 518/719

(57) ABSTRACT

The present invention concerns a catalyst for carrying out hydrocarbon synthesis starting from a mixture comprising carbon monoxide and hydrogen, the active phase of which comprises at least one metal from group VIII deposited on a support formed by at least one oxide, in which said metal from group VIII is selected from the group constituted by cobalt, nickel, ruthenium or iron, and in which said catalyst has an atomic ratio (Co/Al)_{not ground/(CO/Al)}_{ground}, measured by X-ray photo-emission spectroscopy, in the range 1 to 12.

The invention also concerns the catalyst preparation process and its use.

### FIELD OF THE INVENTION

The present invention relates to the field of catalysts used for hydrocarbon synthesis reactions starting from a mixture of gas comprising carbon monoxide and hydrogen, generally termed Fischer-Tropsch synthesis, and more particularly to a catalyst comprising at least one metal from group VIII, preferably cobalt, preferably supported on a silica-alumina.

It is well known to the skilled person that synthesis gas may be converted into hydrocarbons in the present of a catalyst containing metals from group VIII of the periodic classification of the elements such as iron, ruthenium, cobalt or nickel, which catalyze the transformation of a mixture of CO and H₂ known as synthesis gas (i.e. a mixture of carbon monoxide and hydrogen) which may be diluted using carbon dioxide or any other diluent used alone or as a mixture such as methane, nitrogen or ethane, into hydrocarbons which are solid, liquid or gaseous at ambient temperature. That process is known as Fischer-Tropsch synthesis.

Various methods have been described and developed in the prior art in order to improve the preparation of cobalt-based Fischer-Tropsch catalysts supported on various supports. The most widely used are alumina, silica and titanium dioxide, occasionally modified with additional elements. As an example, patent U.S. Pat. No. 6,806,226 describes cobalt-based catalysts. However, the cobalt-based Fischer-Tropsch catalysts which are described therein suffer from the disadvantage of not having a homogenous distribution of cobalt either in the catalyst grains or at the catalyst surface. The poor distribution of cobalt at the surface occurs in the form of agglomeration and cobalt enrichment at the surface and forms a layer which is also known as the (egg)shell. In the case of catalysts shaped for use in a fixed bed, in particular into the form of extrudates or beads, eggshell impregnation may be desirable and thus preferred in order to maximize the accessibility of the reagents to the metal deposited on the surface of the catalytic entity.

Patent U.S. Pat. No. 5,036,032, for example, describes the preparation of a catalyst based on cobalt which can improve the density of active sites on the surface by using a precursor with sufficient viscosity to prevent too deep a penetration into the pores by capillary rise. Direct reduction with no calcining step can increase the dispersion. Silica (SiO₂) is preferred as the support: Thus, diffusional limitations are avoided.

In contrast, in the case of using a catalyst in the form of particles with a grain size of less than 500 μm in slurry bubble column type processes, homogenous distribution and in particular the absence of a shell is generally desirable. Using a slurry process in fact causes high mechanical stress on the catalyst, and so a distribution of the. active metal in the form of a shell renders it more sensitive to attrition effects and may cause a loss of active metal over time. Too great an aggregation of the metal at the catalyst periphery may also cause a loss of selectivity linked to steric constraints (metal crystallites which are aggregated too greatly together), limiting the growth of the hydrocarbon chains and degrading the C5+ selectivity (and as a consequence the chain growth probability, known to the skilled person as alpha) during the hydrocarbon synthesis reaction.

The usual techniques for the preparation of catalysts used for Fischer-Tropsch synthesis generally comprise the following steps: impregnation of the support, drying, calcining and optional reduction.

Hence, several patents describe methods for the preparation of catalysts used in Fischer-Tropsch synthesis based on these usual techniques and intended to improve the homogenous distribution of cobalt in the catalyst and avoid the formation of a shell.

Patent EP 0 736 326 describes a vacuum slurry mode impregnation and two-step drying, a first vacuum step at a temperature in the range 60° C. to 95° C. at the end of which less than 20% of the water remains in the catalyst, and a second step at atmospheric pressure at a temperature in the range 100° C. to 180° C. to eliminate residual water of crystallization.

Patent EP 1 119 411 describes a slurry impregnation step, a two-step vacuum drying step, a first step at a temperature in the range 60° C. to 95° C. and a second step at a higher temperature and lower pressure, followed by calcining at a temperature of 350° C. or less.

Patent U.S. Pat. No. 6,806,226 describes a catalyst obtained by vacuum impregnation and partial vacuum drying at a temperature in the range 60° C. to 95° C. followed by calcining at a temperature in the range 75° C. to 400° C. with a temperature increase rate in the range 0.5° C./min to 1° C./min for hourly space velocities (HSV) of at least 1 m³ of air/(kg Co(NO₃)_{2,}6H₂O×h). That patent envisages the possibility of much more rapid calcining with a temperature increase rate of 100° C./min to eliminate nitrates if the HSV is higher.

The disadvantage of these techniques is that the catalyst still contains water at the end of the drying step since not all of the water supplied during impregnation has been eliminated. The presence of this water will deleteriously affect the homogenous distribution of the cobalt in and at the surface of the catalyst.

The Applicant has observed that surprisingly, effective but not too rapid drying under specific conditions before starting the calcining step can improve the homogeneity of the cobalt in and especially on the surface of the catalyst, completely eliminating the shell of cobalt present at the periphery of the catalyst grains. This means that catalysts with substantially improved activity, C5+ selectivity and paraffin alpha can be obtained.

Thus, the aim of the present invention is to overcome one or more of the disadvantages of the prior art in proposing a catalyst, and a process for its preparation, having improved efficiency due to better distribution of cobalt within the grains and at the surface of the catalyst, To this end, the present invention provides a catalyst for carrying out hydrocarbon synthesis starting from a mixture comprising carbon monoxide and hydrogen, the active phase of which comprises at least one metal from group VIII deposited on a support formed by at least one oxide, in which said metal from group VIII is selected from the group constituted by cobalt, nickel, ruthenium or iron, and said catalyst has an atomic ratio (Co/Al)_{not ground/(Co/Al)}_{ground}, measured by X-ray photo-emission spectroscopy, in the range 1 to 12.

In accordance with one embodiment of the invention, the atomic ratio (Co/Al)_{not ground/(Co/Al)}_{ground} measured by X-ray photo-emission spectroscopy, is in the range 1 to 10.

In accordance with one embodiment of the invention, the atomic ratio (Co/Al) is defined by X-ray photo-emission spectroscopy by determining the measurement for ground particles and for particles which have not been ground, the ratio being equal to: (Co/Al)_{not ground} for particles which have not been ground/(Co/Al)_{ground} for ground particles.

In accordance with one embodiment of the invention, in the ease in which the active phase is constituted by cobalt, nickel or iron, the quantities of cobalt, nickel or iron metals are in the range 1% to 60% by weight and in the case in which the active phase is ruthenium, the quantity of ruthenium is in the range 0.01% to 10% by weight.

In accordance with one embodiment of the invention, the metal from group VIII is cobalt.

In accordance with one embodiment of the invention, the support is formed by at least one simple oxide selected from alumina Al₂O₃, silica SiO₂, titanium oxide TiO₂, ceria CeO₂ and zirconia ZrO₂.

In accordance with one embodiment of the invention, the support is formed by a spinel included in an alumina or a silica-alumina.

In accordance with one embodiment of the invention, the active phase contains at least one additional element selected from the group constituted by ruthenium, molybdenum, tantalum, platinum, palladium and rhenium.

In accordance with one embodiment of the invention, the additional element is ruthenium or rhenium.

The invention also concerns a process for preparing a catalyst as defined above comprising at least one concatenation of the following steps:
a support impregnation step;
a drying step carried out at a temperature of less than 100° C., with a temperature increase rate in the range 0.3° C./min to 1.2° C./min, a gas flow rate in the range 0.5 to 6 Nl/(h.g of catalyst), a pressure equal to 0.1 MPa and for a period in the range 2 h to 15 h;
a calcining step.

In accordance with one implementation of the invention, the concatenation of steps is carried out at least twice.

In accordance with one implementation of the invention, the calcining step is carried out at a temperature in the range 320° C. to 460° C., at a temperature increase rate of 0.3° C./min to 5° C./min and with a gas flow rate in the range 0.3 to 4 Nl of dry air/(h.g of catalyst) and for a period in the range 2 h to 15 h.

The invention also concerns a hydrocarbon synthesis process employing the catalyst described above.

In accordance with one implementation of the invention, the hydrocarbon synthesis is a Fischer-Tropsch synthesis.

In accordance with one implementation of the invention, the hydrocarbon synthesis is carried out in a three-phase reactor in which the catalyst is divided into a particle state with a diameter in the range 5 microns to 300 microns.

### BRIEF DESCRIPTION OF THE DRAWINGS

Other characteristics and advantages of the invention will be better understood and will become clearer from the following description made with reference to the accompanying drawings and given by way of example, in which:

FIG. 1 represents a scanning electron microscope image of a prior art catalyst revealing the presence of a large shell (FIG. 1a) and the aggregation of cobalt within the catalyst grains (FIG. 1b);

FIG. 2 represents a scanning electron microscope image of a prior art catalyst revealing the presence of a large shell and the poor distribution of cobalt within the catalyst grains (aggregates);

FIG. 3 represents a scanning electron microscope image of the catalyst of the invention revealing the presence of a slight shell (FIG. 3a) and slight aggregation of cobalt within the catalyst grains (FIG. 3b).

The catalyst of the invention may be used in reactions carried out in suspension in a three-phase fluidized reactor, preferably of the bubble column type. In this preferred implementation of the catalyst, the catalyst is divided into a very fine powder state, particularly of the order of a few tens of microns, for example in the range 5 microns to 300 microns, preferably in the range 20 microns to 150 microns, and more preferably in the range 20 to 120 microns. This technique is also known to the skilled person as a “slurry” process.

The catalyst of the invention May also be used in different types of reactors, for example in a fixed bed, moving bed, ebullated bed or three-phase fluidized bed.

The catalyst of the invention comprises a metallic active phase deposited on a support. The active phase comprises at least one metal from group VIII preferably selected from cobalt, nickel, ruthenium and iron. In the case in which the active phase comprises at least one metal selected from cobalt, nickel and iron, its quantity represents 1% to 60% by weight with respect to the weight of catalyst, preferably 5% to 30% by weight with respect to the weight of the catalyst and more preferably 10% to 30% by weight with respect to the weight of the catalyst. In the case in which the active phase comprises ruthenium, the quantity of ruthenium is in the range 0.01% to 10% by weight with respect to the weight of catalyst, highly preferably in the range 0.05% to 5% by weight with respect to the weight of the catalyst. The active phase deposited on the support may comprise one or more metals selected from cobalt, nickel, ruthenium and iron. Highly preferably, the active phase comprises cobalt. The active phase of the catalyst also advantageously comprises at least one additional metal selected from platinum, palladium, rhenium, ruthenium, manganese and tantalum and highly preferably selected from platinum, ruthenium and rhenium. The additional metal(s) is (are) preferably present in a quantity of 0.01% to 2% by weight, preferably 0.03% to 0.5% by weight with respect to the Weight of the catalyst.

The catalyst of the invention is in the oxide form before activation thereof, which consists of reduction. It has crystallites of the oxide of the metal from group VIII present in the active phase of the catalyst, preferably crystallites of cobalt oxide, Co₃O₄.

The support on which the active phase is deposited is advantageously formed from at least one simple oxide selected from alumina (Al₂O₃), silica (SiO₂), titanium oxide (TiO₂), ceria (CeO₂) and zirconia (ZrO₂). It may also advantageously be formed by a plurality of simple oxides selected from alumina (Al₂O₃), silica(SiO₂), titanium oxide (TiO₂), ceria (CeO₂) and zirconia (ZrO₂). Highly preferably, the support used for the catalyst of the invention is formed from silica and alumina. This support constituted by silica-alumina preferably comprises 1% to 30% by weight of silica. The silica-alumina is homogenous on the micrometric scale, and still more preferably homogenous on the nanometric scale. The support on which the active phase is deposited may also advantageously be formed by a spinel included in an alumina or silica-alumina, preferably in a silica-alumina. In particular, the catalyst support may advantageously be constituted by a simple spinel, included in a silica-alumina, of the type MAl₂O₄/Al₂O₃.SiO₂ or a mixed spinel included in a silica-alumina, of the type MₓM′₍₁₋ₓ₎AL₂O₄/Al₂O₃.SiO₂, where M and M′ are distinct metals selected from the group constituted by magnesium (Mg), copper (Cu), cobalt (Co), nickel (Ni), tin (Sn), zinc (Zn), lithium (Li), calcium (Ca), caesium (Cs), sodium (Na), iron (Fe) and manganese (Mn), where Al₂O₃.SiO₂ designates the chemical formula for a silica-alumina, where x is in the range 0 to 1, the values 0 and 1 being excluded. Such a support formed from a spinel structure comprises at least 5% by weight of said spinel structure, preferably at least 10% by weight, and still more preferably at least 15% by weight. The silica-alumina in which the spinel structure is preferably included preferably comprises 1% to 30% by weight of silica. It is homogenous on the micrometric scale, and still more preferably homogenous on the nanometric scale.

A support formed with a spinel included in an alumina or a silica-alumina may be prepared using any method known to the skilled person. In particular, any process that can produce the support modified by the addition of at least one element M to obtain a simple spinel structure and at least one element M′ to obtain a mixed spinel structure is suitable. One method for preparing such a support consists, for example, in impregnating an alumina or silica-alumina support, preformed or in powder form, with at least one aqueous solution containing hydrosoluble precursors of the elements selected for the spinel structure and then in general carrying out washing, drying and finally calcining steps. Another method consists of preparing the catalyst support based on alumina or silica-alumina by co-precipitation of an aqueous solution containing the metals Al, M and optionally M′, for example in the form of nitrates, with an aqueous solution of an alkali carbonate or bicarbonate, followed by washing, drying, and finally calcining. Yet another method consists of preparing the catalyst support based on alumina or silica-alumina using the sol-gel process or by complexing an aqueous solution containing the metals Al, M and optionally M′ with at least one alcohol alpha acid added in an amount of 0.5 to 2 moles of acid per mole of Metals, followed by vacuum drying to produce a homogenous vitreous substance, then by calcining. Said support undergoes heat treatment before the preparation proper of the catalyst used in the process of the invention in order to obtain the spinel structure (aluminate of M or aluminate of M and M′ where M and M′ have the definitions given above). The heat treatment is preferably carried out at a temperature in the range 600° C. to 1200° C., highly preferably in the range 740° C. to 1030° C. and still more preferably in the range 800° C. to 980° C. It is carried out in an oxidizing atmosphere, for example in air or in oxygen-depleted air. It may also be at least partially carried out in nitrogen.

The support onto which the active phase is deposited may have a morphology which is in the form of a powder with a variable granulometry, especially when the catalyst is used in a slurry bubble column. The grain size of the catalyst may be in the range from a few microns to a few hundred microns. For use in a “slurry” reactor, the particle size of the catalyst is preferably in the range 10 microns to 500 microns, more preferably in the range 10 microns to 300 microns, highly preferably in the range 20 to 1.50 microns, and still more preferably in the range 30 to 120 microns.

The catalyst of the invention. is prepared using a process comprising at least:
a solution impregnation step;
a drying step;
a calcining step.

The steps for impregnation, drying and calcining are carried out at least once. In accordance with a preferred implementation of the invention, the concatenation of the steps of impregnation, drying and calcining in that order is carried out at least twice.

The catalyst preparation process may also include a step for stabilizing the support carried out before the support impregnation step.

The catalyst preparation process of the invention may also include a step for reduction of the catalyst, carried out after the concatenation of the impregnation, drying and calcining steps.

The Applicant has discovered that highly efficient and complete drying of the catalyst before the calcining step prevents the formation of a shell of metal from group VIII, especially cobalt, on the surface of the catalyst particles and also improves the distribution of the metal from group VIII, especially cobalt, within the catalyst particles.

After each impregnation step, the catalyst preparation process of the invention thus comprises a drying step independent of the calcining step, which can avoid migration of the metal from group VIII, in particular cobalt, to the surface, and also avoids the formation of aggregates of metal from group VIII, especially cobalt.

### Stabilization of the Support

The support is stabilized by dry impregnation of the silica-alumina using an aqueous solution of a salt of a metal from group VIII, especially cobalt, the .impregnated solid then being dried at a temperature in the range 60° C. to 200° C. for a period of half an hour to three hours, then calcined at a temperature in the range 300° C. to 600° C. in dry air for a period of half an hour to three hours, then at a temperature in the range 700° C. to 1100° C. for a period of one hour to 24 hours, preferably 2 hours to 5 hours, the solid obtained containing at least 5% by weight of spinel structure comprising cobalt, preferably at least 10% by weight and still more preferably at least 15% by weight. This stabilization means that attacks by the Fischer-Tropsch synthesis reaction mixture (water, acid) can be limited. The metal from group VIII, especially cobalt, which is then added has a very strong interaction with the support and thus cannot be reduced.

### Impregnation of Support

The support or stabilized support is impregnated using at least one solution containing at least one precursor of said metal from group VIII. In particular, this step may be carried out by dry impregnation, excess impregnation or by deposition-precipitation using methods which are well known to the skilled person. Preferably, this impregnation step is carried out by dry impregnation at ambient temperature, i.e. at a temperature of 20° C., which consists of bringing the catalyst support into contact with a solution containing at least one precursor of said metal from group VIII wherein the volume is equal to the pore volume of the support to be impregnated. This impregnation may be carried out at any other temperature which is compatible with this technique, for example in the range 5° C. to 40° C., preferably in the range 15° C. to 25° C., and more preferably in the range 17° C. to 23° C. This solution contains metallic precursors of the metal or Metals from group VIII in the desired concentration in order to obtain the planned quantity of metal on the final catalyst.

The metal or metals from group VIII are brought into contact with the support by means of any metallic precursor which is soluble in aqueous phase or in an organic phase. When it is introduced in organic solution, the precursor of the metal from group VIII is, for example, the oxalate or acetate of said metal from group VIII. Preferably, the precursor of the metal from group VIII is introduced in aqueous solution, for example in the form of nitrate, carbonate, acetate, chloride, oxalate, complexes formed using a polyacid or an acid-alcohol and its salts, complexes formed with acetylacetonates, or any other inorganic derivative which is soluble in aqueous solution, which is brought into contact with said support. In the preferred case where the metal from group VIII is cobalt, the precursor used is advantageously cobalt, cobalt nitrate, cobalt oxalate or cobalt acetate.

The preparation of the catalyst of the invention advantageously comprises at least one supplemental step consisting of depositing at least one additional metal selected from platinum, palladium, rhenium, rhodium, ruthenium, manganese and tantalum onto said catalyst support. Preferably, the additional metal is selected from platinum, ruthenium and rhenium and highly preferably, the additional metal is platinum. The additional metal may be deposited on the support using any method known to the skilled person, preferably by impregnation of the catalyst support using at least one solution containing at least one precursor of said additional metal, for example by dry impregnation or by excess impregnation.

### Drying

Drying is an important step in the preparation of the catalyst of the invention. The steps of impregnation, drying and calcining are carried out independently of each other.

Drying carried out for the preparation of the catalyst of the invention may be carried out in a tube furnace, in an oven in the presence of a flow of gas, preferably dry air, and in general in any type of vessel known to the skilled person that can be used to circulate air at a high flow rate. In the case in which drying is carried out in a tube furnace or in an oven or any other equipment of the same type, it is carried out at a temperature of less than 100° C., preferably 80° C. or less, and highly preferably in the range 40° C. to 80° C. The temperature increase rate used to reach the operating temperature, which is ambient temperature, generally in the range 15° C. to 25° C., to a temperature of less than 100° C., preferably 80° C. or less and highly preferably in the range 40° C. to 80° C., is in the range 0.1° C./min to 3.6° C./min, preferably in the range 0.3° C./min to 1.2° C./min. This drying step lasts for a period in the range 2 h to 15 h, preferably in the range 4 h to 12 h. The gas flow rate employed is in the range 0.5 to 6 Nl/(h.g of catalyst) (normal litres per hour per grain of catalyst), preferably in the range 1 to 4 Nl/(h.g of catalyst).

Drying is carried out at atmospheric pressure, i.e. 0.1 MPa.

### Calcining

The calcining step is carried out at a temperature in the range 320° C. to 460° C., preferably in the range 350° C. to 440° C. In the case in which drying is carried out in a tube furnace or oven, the calcining is carried out in the same vessel. At the end of the drying step at a temperature below 100° C., preferably 80° C. or less, the temperature is increased at an increase rate of 0.1° C./min to 10° C./min and preferably 0.3° C./min to 5° C./min and at a flow rate in the range 0.3 to 4 Nl of dry air/(h.g of catalyst), preferably in the range 0.5 to 3 Nl(h.g of catalyst), and highly preferably in the range 0.6 to 0.9 Nl/(h.g of catalyst).

It is preferably carried out for a period in the range 2 h to 15 h, preferably in the range 4 h to 12 h.

Catalyst reduction

Prior to using it in a catalytic reactor, the catalyst undergoes at least one reducing treatment, for example in hydrogen, pure or diluted, at high temperature. This treatment can activate said catalyst and form particles of metal, in particular metal from group VIII, with a zero valent state. The temperature of said reduction treatment is preferably in the range 200° C. to 500° C. and its duration is in the range 2 to 20 hours.

This reduction treatment is carried out either in situ (in the same reactor as that where the Fischer-Tropsch reaction is carried out) or ex situ before being loaded into the reactor. When the metal from group VIII used is cobalt, the reduction step is used to carry out the following reaction:

Co₃O₄→CoO→Co(0)

Detection of Layer of Metal from Group VIII on Surface of Catalyst Particles

Usually, the distribution of cobalt within the catalyst particles as well as the presence or absence of a layer of metal from group VIII, especially cobalt, also known as the shell, is detected by electron probe X-ray microanalysis. Electron probe X-ray microanalysis has a spatial resolution of the order of a. few microns. A shell with a thickness of less than a micron could not be detected by this technique. The distribution of cobalt within the catalyst grains and the shell may also be visualized by scanning electron microscopy (SEM). However, in cases where the shell thickness is irregular or less than 20 nm, this technique cannot reveal the shell even if one is present. Further, it cannot completely resolve the distribution of the cobalt (aggregates).

Surprisingly, the Applicant has observed that an analysis carried out on samples prepared in suitable manner using the. X-ray photoemission spectrometry technique (XPS) can reveal a shell which is not visible in scanning electron microscopy. Thus, XPS can reveal differences in homogeneity between two preparations which are observed to be homogenous using SEM. XPS can also show that certain specific preparations of the catalyst may result in a distribution which is more homogenous than others, while SEM would have shown them to be equivalent.

In order to refine the measurement of the shell and the distribution of the cobalt, the X-ray photoemission spectrometry technique is thus used in the context of the invention. In this technique, the catalyst particles are irradiated by monochromatic X-rays which cause the ionization of atoms by a photo-electric effect. The kinetic energy E_{c} of those photoelectrons is measured, producing the spectrum of the intensity of the electrons as a function of the energy measured.

Each incident X-ray photon has the same energy hν, since the beam is monochromatic (h being Planck's constant and ν the frequency of the incident light wave). During interaction with the atom, a portion of that energy serves to extract one or more electrons bound to its atomic orbital by a binding energy termed. E_{L}; the remainder is transferred to the electron in the form of kinetic energy.

The kinetic energy spectrum thus has peaks and the binding energy corresponding to each peak can be determined using the following relationship:

E_{L}=h×ν−E_{c}

where E_{L}=binding energy [J]
E_{c}=electron kinetic energy [J]
h=Planck's constant [J s](˜6.626 0 755×10⁻³⁴)
ν=frequency of radiation [s⁻¹]

The energy of the incident X-ray photon is of the same order of magnitude as the ionization energy of the core electrons: their emission produces XPS. peaks which thus are essentially characteristic of the nature of the atom; on the other hand, chemical information (especially the oxidation number) is obtained from the small displacements of the XPS peak corresponding to the variation in energy between valency layers, this latter (corresponding in general to the UV/visible/near IR region) is small compared with that of X-rays.

Thus, it is possible to obtain access to the chemical composition of the outer surface of the material analyzed over a depth of approximately 5 nanometres (which corresponds to the mean free path of X-ray photons) by comparison with known spectra.

XPS measurement of the atomic ratio of cobalt to alumina (Co/Al) is carried out on the one hand on ground catalyst grains and on the other hand on grains which have not been ground. If the distribution of the metal is homogenous and there is thus no surface shell, then the (Co/Al)_{ground} atomic ratio obtained for the ground catalyst and the (Co/Al)_{not ground} atomic ratio obtained for the catalyst which has not been ground should be identical and the ratio of the two should tend towards 1.

It is thus possible to characterize the catalyst of the invention by determining the atomic ratio (Co/Al) by XPS (Co/Al)/XPS) of the catalyst which has not been ground ((Co/Al)_{not ground} which is termed NG to simplify the formula) and the ground catalyst ((Co/Al)_{ground}, termed G to simplify the formula) and to calculate the (Co/Al)_{XPS} ratio of the 2 results. The atomic ratio measured using XPS can be written as follows:

(Co/Al)_{XPS}=(Co/Al)_{not ground}/(CO/Al)_{ground}=NG/G.

The thinner the shell, the nearer the ratio tends to 1. The catalyst of the invention used for Fischer-Tropsch synthesis has a (CO/Al)_{not ground}/(CO/Al)_{ground} atomic ratio or NG/G in the range 1 to 20, in the range 1 to 15, preferably in the range 1 to 12, and more preferably in the range 1 to 10, and still more preferably in the range 1 to 5.

In a variation of the invention, the (Co/Al)_{not ground}/(Co/Al)_{ground} atomic ratio or NB/B may be in the range 1 to 5, in the range 5 to 10, in the range 10 to 12, in the range 10 to 15 or in the range 15 to 20 depending on whether a shell is present or not and its thickness.

The invention will be illustrated by the following examples.

### EXAMPLES

### Comparative Example 1 (Standard)

A catalyst A1 comprising cobalt deposited on a silica-alumina support was prepared by dry impregnation of an aqueous solution of cobalt nitrate in order to deposit of the order of 13.5% by weight of Co in two successive steps on a commercial silica-alumina powder (SIRALOX® 5/170, SASOL) with a mean grain size of 80 μm, a surface area of 171 m²/g and a pore volume equal to 0.519 ml/g.

After a first dry impregnation, the solid was oven dried at 120° C. for 3 h in a flow of air then calcined at 420° C. for 4 h in an oven in a flow of air with a non-controlled flow rate. The intermediate catalyst contained approximately 8% by weight of Co. It underwent a second dry impregnation using a cobalt nitrate solution. The solid obtained was oven dried at 120° C. for 3 h in a flow of air with a non-controlled flow rate then calcined at 420° C. for 4 h in an oven in a flow of air. The final catalyst A1 was obtained; it contained 13.5% by weight of Co.

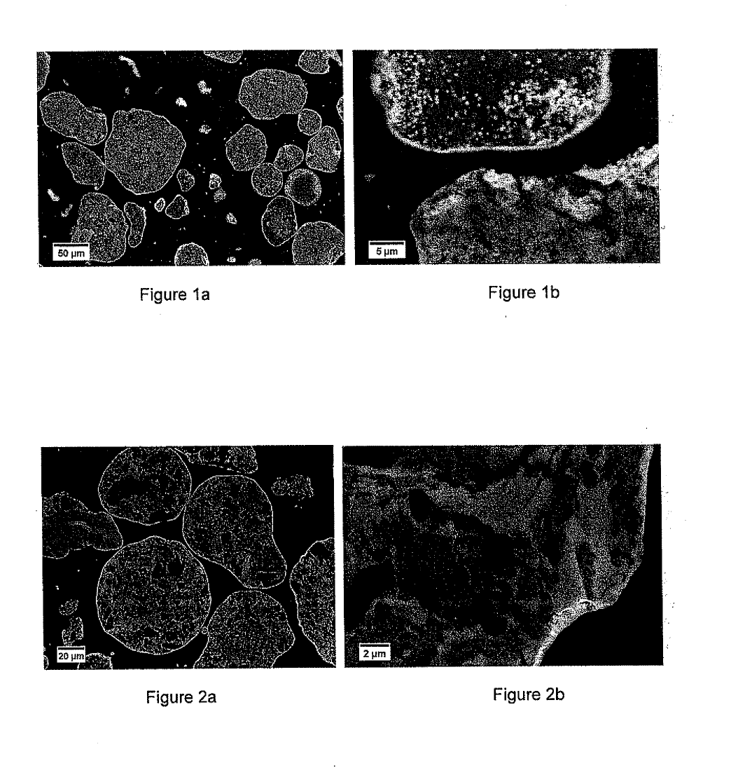

Scanning electron microscopy (FIG. 1) revealed the presence of shell (FIG. 1a) and a poor distribution of cobalt within the catalyst grains (FIG. 1b).

The XPS measurement produced a ratio, as defined in the text, of 18.6. This value confirmed the presence of the shell observed with the scanning electron microscope (FIG. 1).

### Comparative Example 2 (Standard)

A catalyst A2 comprising cobalt deposited on a silica-alumina support was prepared by dry impregnation of an aqueous solution of cobalt nitrate in order to deposit of the order of 13.5% by weight of Co in two successive steps on a commercial silica-alumina powder (SIRALOX® 5/170, SASOL) with a mean grain size of 80 μm, a surface area of 171 m²/g and a pore volume equal to 0.519 ml/g.

After a first dry impregnation, the solid was dried in a tube furnace in a flow of dry air from 20° C. to 120° C. at a temperature increase rate of 0.3° C./min in a flow of air of 12 Nl/(h.g of catalyst), the catalyst was kept at 120° C. for 4 h in a flow of air then, without being discharged, the temperature was raised to 420° C. at an increase rate of 1° C./min and in a flow or air of 12 Nl/h for 4 h. The intermediate catalyst contained approximately 8% by weight of Co. It underwent a second dry impregnation using a cobalt nitrate solution. The moist solid obtained was dried in a tube furnace in a flow of dry air from 20° C. to 120° C. at a rate of increase of 0.3° C./min in a flow of air of 12 Nl/(h.g of catalyst), the catalyst was kept at 120° C. for 4 h in a flow of air then, without being discharged, the temperature was raised to 420° C. at a rate of increase of 1° C./min and in a 12 Nl/h flow of air for 4 h. The final catalyst A2 was obtained; it contained 13.5% by weight of Co.

The XPS measurement produced a ratio, as defined in the text, of 13.2. This value confirmed the presence of the shell observed with the scanning electron microscope (FIG. 2).

### Comparative Example 3 (According to the Invention)

A catalyst A3 comprising cobalt deposited on a silica-alumina support was prepared by dry impregnation of an aqueous solution of cobalt nitrate in order to deposit of the order of 13.5% by weight of Co in two successive steps on a commercial silica-alumina powder (SIRALOX® 5/170, SASOL) with a mean grain size of 80 μm, a surface area of 171 m²/g and a pore volume equal to 0.519 ml/g.

After a first dry impregnation, the solid was dried in a tube furnace in a flow of dry air from ambient temperature to 80° C. at a temperature increase rate of 0.3° C./min in a flow of air of 1 Nl/h/g of catalyst, the catalyst was kept at 80° C. for 5 h in a flow of air then, without being discharged, the temperature was raised to 360° C. at an increase rate of 1° C./min and in a flow or air of 0.5 Nl/(h.g of catalyst) for 4 h. The intermediate catalyst contained approximately 8% by weight of Co. It underwent a second dry impregnation using a cobalt nitrate solution. The moist solid obtained was dried in a tube furnace in a flow of dry air from ambient temperature to 80° C. at a rate of 0.3° C./min in a flow of air of 1 Nl/h/g of catalyst, the catalyst was kept at 80° C. for 5 h in a flow of air then, without being discharged, the temperature was raised to 360° C. at a rate of 1° C./min and in a 0.5 Nl/(h.g of catalyst) flow of air for 4 h. The final catalyst A3 was obtained; it contained 13.5% by weight of Co.

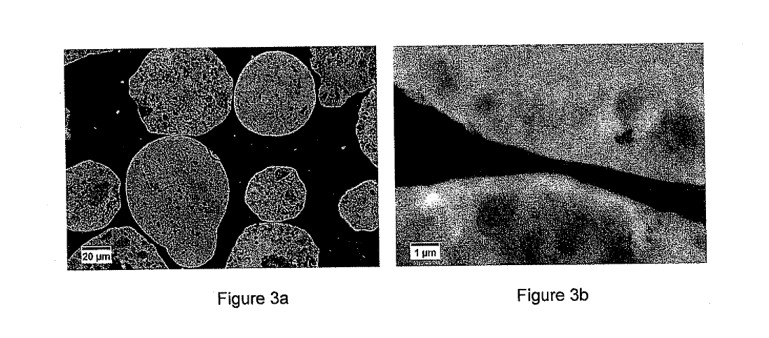

Scanning electron microscopy (FIG. 3) revealed the absence of a regular shell (FIG. 3a) and good distribution of cobalt within the catalyst grains (FIG. 3b),

The XPS measurement produced a ratio, as defined in the text, of 10.8. This value confirmed the presence of a very small shell which could not be observed with the scanning electron microscope (FIG. 3).

TABLE 1
Summary of Examples 1, 2 and 3

| Catalyst | (Co/A1)_{XPS} | SEM shell |
|---|---|---|
| A1 | 18.6 | Large |
| A2 | 13.2 | Weak |
| A3 | 10.8 | none |

### Example 4: Catalytic Performances of Catalysts A1, A2 and A3 in Synthesis Gas Conversion

Before being tested in succession in synthesis gas conversion, catalysts A1, A2 and A3 were reduced ex situ in a flow of pure hydrogen at 400° C. for 16 hours in a tube reactor. Once the catalyst had been reduced, it was discharged in an argon atmosphere and coated with Sasolwax® then stored prior to test. The Fischer-Tropsch synthesis reaction was carried out in a continuously functioning slurry type reactor operating at a concentration of 10% (vol) of catalyst in the slurry phase.

Each of the catalysts was in the powder form with a diameter in the range 40 to 150 microns.

The test conditions were as follows:
Temperature=220° C.
Total pressure=2 MPa
H₂/CO molar ratio 32 2/1
Hourly space velocity (HSV)=1000 h⁻¹

The test conditions were adjusted for identical CO conversions irrespective of the activity of the catalyst.

The activity is calculated as follows for a reference catalyst; the reference temperature is equal to the base temperature, and thus the activity is 100%.
Activity=exp(—E/RT_{base})exp(E/RT_{ref})
Where T=225° C.
   E=activation energy
   R=perfect gas constant

The results were calculated for catalysts A1, A2 and A3 with respect to the reference catalyst with 100% activity and are shown in Table 2. The alpha paraffin selectivities are also given, as well as the selectivity for C5+ compounds, and the CH₄ selectivity.

TABLE 2

| | | | Alpha | CH₄ |
|---|---|---|---|---|
| | Activity after | C5+ | paraffin | selec- |
| Catalyst | 300 h of test (%) | selectivity | selectivities | tivity |
| A1 | 290 | 80 | 0.900 | 87 |
| (comparative) | | | | |
| A2 | 490 | 83 | 0.880 | 7.9 |
| (comparative) | | | | |
| A3 (invention) | 450 | 85 | 0.905 | 7.4 |

The activity of the catalyst of the invention is improved compared with the activity of catalyst A1 (450 compared with 290). Its C5+ selectivity, alpha paraffin selectivity was also better than that of catalyst A1 and catalyst A2. In surprising manner, the process of the invention can thus be used to obtain a catalyst with very little shell (not visible in SEM but just detectable in XPS) and is both more active and more selective. Catalyst A3 of the invention can also be used to limit the CH₄ selectivity compared with catalyst A1 and A2.

The alpha paraffin selectivity was measured by as chromatography carried out on the reaction effluents, assay of the paraffins and calculating the slope of the log mol (%)=f(number of carbons) curve which corresponds to alpha.

The reduction in the CH₄ selectivity is also a substantial improvement in the catalyst, since chain growth should be encouraged, thereby limiting the loss of carbon caused by the formation of short molecules.

The present invention is not limited to the details given above; embodiments are allowable using many other specific forms without departing from the field of application of the invention. As a consequence, the present embodiments should be considered to be by way of illustration and may be modified without, however, departing from the scope defined by the claims.

Without further elaboration, it is believed that one skilled in the art can, using the preceding description, utilize the present invention to its fullest extent. The preceding preferred specific embodiments are, therefore, to be construed as merely illustrative, and not limitative of the remainder of the disclosure in any way whatsoever.

The entire disclosures of all applications, patents and publications, cited herein and of corresponding FR application No. 10/03.006, filed Jul. 16, 2010, are incorporated by reference herein.

From the foregoing description, one skilled in the art can easily ascertain the essential characteristics of this invention and, without departing from the spirit and scope thereof, can make various changes and modifications of the invention to adapt it to various usages and conditions.

1. A catalyst for carrying out hydrocarbon synthesis starting from a mixture comprising carbon monoxide and hydrogen, the active phase of which comprises at least one metal from group VIII deposited on a support formed by at least one oxide, in which said metal from group VIII is selected from the group constituted by cobalt, nickel, ruthenium or iron, and in which said catalyst has an atomic ratio (Co/Al)_{not ground}/(Co/Al)_{ground}, measured by X-ray photo-emission spectroscopy, in the range 1 to 12.

2. A catalyst according to claim 1, in which the atomic ratio (Co/Al)_{not ground}/(Co/Al)_{ground}, measured by X-ray photo-emission spectroscopy, is in the range 1 to 10.

3. A catalyst according to claim 1, in which the atomic ratio (Co/Al) is defined by X-ray photo-emission spectroscopy by determining the measurement for ground particles and for particles which have not been ground, the ratio being equal to: (Co/Al)_{not ground} for particles which have not been ground/(Co/Al)_{ground} for ground particles.

4. A catalyst according to claim 1 in which, in the case in which the active phase is constituted by cobalt, nickel or iron, the quantities of cobalt, nickel or iron metals are in the range 1% to 60% by weight and in the case in which the active phase is ruthenium, the quantity of ruthenium is in the range 0.01% to 10% by weight.

5. A catalyst according to claim 1, in which the metal from group VIII is cobalt.

6. A catalyst according to claim 1, in which the support is formed by at least one simple oxide selected from alumina Al₂O₃, silica SiO₂, titanium oxide TiO₂, ceria CeO₂ and zirconia ZrO₂.

7. A catalyst according to claim 1, in which the support is formed by a spinel included in an alumina or a silica-alumina.

8. A catalyst according to claim 1, in which the active phase contains at least one additional element selected from the group constituted by ruthenium, molybdenum, tantalum, platinum, palladium and rhenium.

9. A catalyst according to claim 8, in which said additional element is ruthenium or rhenium.

10. A process for preparing a catalyst as defined in claim 1 comprising at least one concatenation of the following steps:
a support impregnation step;
a drying step carried out at a temperature of less than 100° C., with a temperature increase rate in the range 0.3° C./min to 1.2° C./min, a gas flow rate in the range 0.5 to 6 Nl of dry air/(h.g of catalyst), a pressure equal to 0.1 MPa and for a period in the range 2 h to 15 h;
a calcining step.

11. A process according to claim 10, in which the concatenation of the impregnation, drying and calcining steps is carried out at least twice.

12. A process according to claim 10, in which the calcining step is carried out at a temperature in the range 320° C. to 460° C., at a temperature increase rate of 0.3° C./min to 5° C./min and with a gas flow rate in the range 0.3 to 4 Nl of dry air/(h.g of catalyst) and for a period in the range 2 h to 15 h.

13. A process for synthesizing hydrocarbons employing a catalyst according to claim 1.

14. A process according to claim 13, in which the hydrocarbon synthesis is a Fischer-Tropsch synthesis.

15. A process according to claim 13, in which the hydrocarbon synthesis is carried out in a three-phase reactor in which the catalyst is divided into a particle state with a diameter in the range 5 microns to 300 microns.
